(19) United States
(12) Patent Application Publication
(10) Pub. No.: US 2009/0131786 A1
(43) Pub. Date: May 21, 2009

(54) ULTRASONIC DIAGNOSTIC APPARATUS AND ULTRASONIC DIAGNOSTIC APPARATUS SYSTEM

(75) Inventors: Shinichi Amemiya, Tokyo (JP); Koji Yanagihara, Tokyo (JP); Dai Morita, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(73) Assignee: GE MEDICAL SYSTEMS GLOBAL TECHNOLOGY COMPANY, LLC, Waukesha, WI (US)

(21) Appl. No.: 12/267,155

(22) Filed: Nov. 7, 2008

(30) Foreign Application Priority Data
Nov. 9, 2007 (JP) 2007-291654

(51) Int. Cl. A61B 8/00

(52) U.S. Cl. 600/437

(57) ABSTRACT

An ultrasonic diagnostic apparatus includes an ultrasonic diagnostic apparatus body having a console interface device that permits changeable connection of a plurality of consoles of different types, a console identifying device that identifies the type of the connected console and an operation mode control device that so performs control as to accomplish operation in the diagnostic mode corresponding to the identified console, and a console of at least one type of the plurality of consoles of different types.

### CROSS REFERENCE TO RELATED APPLICATIONS

This application claims the benefit of Japanese Patent Application No. 2007-291654 filed Nov. 9, 2007, which is hereby incorporated by reference in its entirety.

### BACKGROUND OF THE INVENTION

The subject matter disclosed herein relates to an ultrasonic diagnostic apparatus and an ultrasonic diagnostic apparatus system, and in more detail relates to an ultrasonic diagnostic apparatus and an ultrasonic diagnostic apparatus system capable of executing a plurality of functions required in hospital with a single apparatus and permitting the use of a convenient-to-use console.

Conventionally, ultrasonic diagnostic apparatuses enabled to operate in a functionally extended operation mode by adding extended function modules for a dedicated abdominal function, a dedicated cardiac function, a dedicated obstetric/gynecological function, a Doppler mode image generating function, a power mode image generating function, a color flow image generating function and so forth are known (see Japanese Unexamined Patent Publication No. 2003-079628 for instance).

Ultrasonic diagnostic apparatuses specialized in operations for ultrasonic diagnosis of circulatory organs are also known (see Japanese Unexamined Patent Publication No. 2002-272739 and Japanese Unexamined Patent Publication No. 2007-175069 for instance).

Furthermore, ultrasonic diagnostic apparatuses specialized in operations for ultrasonic diagnosis of fetuses are also known (see Japanese Unexamined Patent Publication No. Hei 7 (1995)-116159 for instance).

Ultrasonic diagnostic apparatuses enabled to operate in a functionally extended operation mode by adding extended function modules are superior in cost effectiveness because they can execute a plurality of functions required in hospital with a single apparatus.

However, they necessitate a console adaptable to a plurality of extended functions, but such a console would have arrays of buttons and keys that are never used during operation with one function, and therefore involves a problem of inconvenience in using the console.

On the other hand, an ultrasonic diagnostic apparatus specialized in operations for a single function would have on its console no array of buttons and the like which are never used, and accordingly the console would be more convenient to use.

However, this would necessitate ultrasonic diagnostic apparatuses each specialized in operations for one or another of a plurality of functions required in hospital, and accordingly might entail a problem of poorer cost effectiveness.

### BRIEF DESCRIPTION OF THE INVENTION

It is desirable that the problems described previously are solved.

In a first aspect, an ultrasonic diagnostic apparatus includes an ultrasonic diagnostic apparatus body having a console interface device that permits changeable connection of a plurality of consoles of different types, a console identifying device that identifies the type of the connected console and an operation mode control device that so performs control as to accomplish operation in the diagnostic mode corresponding to the identified console; and a console of at least one type of the plurality of consoles of different types.

The ultrasonic diagnostic apparatus according to the first aspect excels in cost effectiveness because a plurality of functions required in hospital can be executed with a single apparatus by changing the type of the console connected to the ultrasonic diagnostic apparatus body. And as it permits the use of a console specialized in the operation for each of different functions, it involves no array of buttons or the like which are never used on the console, and accordingly the console would be more convenient to use.

According to a second aspect, the ultrasonic diagnostic apparatus according to the first aspect, is characterized in that the type of the console differs with the object of diagnosis.

The ultrasonic diagnostic apparatus according to the second aspect enables the type of the console connected to the ultrasonic diagnostic apparatus body to be changed according to the object of diagnosis.

According to a third aspect, the ultrasonic diagnostic apparatus according to the second aspect, is characterized in that the objects of diagnosis are liver/kidney and heart.

The ultrasonic diagnostic apparatus according to the third aspect enables the type of the console connected to the ultrasonic diagnostic apparatus body to be changed according to whether the object of diagnosis is the liver/kidney or the heart.

According to a fourth aspect, the ultrasonic diagnostic apparatus according to the second aspect, is characterized in that the objects of diagnosis are the liver/kidney and the fetus.

The ultrasonic diagnostic apparatus according to the fourth aspect enables the type of the console connected to the ultrasonic diagnostic apparatus body to be changed according to whether the object of diagnosis is the liver/kidney or the fetus.

According to a fifth aspect, the ultrasonic diagnostic apparatus according to the second aspect, is characterized in that the objects of diagnosis are the liver/kidney, the heart and the fetus.

The ultrasonic diagnostic apparatus according to the fifth aspect enables the type of the console connected to the ultrasonic diagnostic apparatus body to be changed according to whether the object of diagnosis is the liver/kidney, the heart or the fetus.

According to a sixth aspect, the ultrasonic diagnostic apparatus according to any of the first through fifth aspects, is characterized in that the console is equipped with a wireless communication interface for wireless communication with the ultrasonic diagnostic apparatus body and the ultrasonic diagnostic apparatus body is equipped with a wireless communication interface for wireless communication with the console.

As the ultrasonic diagnostic apparatus according to the sixth aspect is wirelessly connected, there is no need to engage or disengage connectors, and accordingly the console can be replaced quickly.

According to a seventh aspect, the ultrasonic diagnostic apparatus according to the first aspect, is characterized in that the type of the console differs with the clinical department.

The ultrasonic diagnostic apparatus according to the seventh aspect enables the type of the console connected to the ultrasonic diagnostic apparatus body to be changed according to the clinical department.

According to an eighth aspect, the ultrasonic diagnostic apparatus according to the seventh aspect, is characterized in that the clinical departments are the internal medicine department/urological department and the cardiovascular department.

The ultrasonic diagnostic apparatus according to the eighth aspect enables the type of the console connected to the ultrasonic diagnostic apparatus body to be changed according to whether the clinical department is the internal medicine department/urological department or the cardiovascular department.

According to a ninth aspect, the ultrasonic diagnostic apparatus according to the seventh aspect, is characterized in that the clinical departments are the internal medicine department/urological department and the obstetrics department/gynecology department.

The ultrasonic diagnostic apparatus according to the ninth aspect enables the type of the console connected to the ultrasonic diagnostic apparatus body to be changed according to whether the clinical department is the internal medicine department/urological department or the obstetrics department/gynecology department.

According to a tenth aspect, the ultrasonic diagnostic apparatus according to the seventh aspect, is characterized in that the clinical departments are the internal medicine department/urological department, the cardiovascular department and the obstetrics department/gynecology department.

The ultrasonic diagnostic apparatus according to the tenth aspect enables the type of the console connected to the ultrasonic diagnostic apparatus body to be changed according to whether the clinical department is the internal medicine department/urological department, the cardiovascular department or the obstetrics department/gynecology department.

According to an eleventh aspect, the ultrasonic diagnostic apparatus according to any of the seventh through tenth aspects, is characterized in that the console is equipped with a wireless communication interface for wireless communication with the ultrasonic diagnostic apparatus body and the ultrasonic diagnostic apparatus body is equipped with a wireless communication interface for wireless communication with the console.

As the ultrasonic diagnostic apparatus according to the eleventh aspect is wirelessly connected, there is no need to engage or disengage connectors, and accordingly the console can be replaced quickly.

According to a twelfth aspect, an ultrasonic diagnostic apparatus system includes N (≧3) ultrasonic diagnostic apparatus bodies each having a console interface device that permits changeable connection of a plurality of consoles of different types, a console identifying device that identifies the type of the connected console and an operation mode control device that so performs control as to accomplish operation in the diagnostic mode corresponding to the identified console; and M (>N) consoles of at least two types of the plurality of consoles of different types.

The ultrasonic diagnostic apparatus system according to the twelfth aspect enables the system to be flexibly altered, such as, where N=3, M=4, two consoles of a first type and two consoles of a second type are available, using two first type console and one second type console if the number of patients to be diagnosed with the first type is greater or using one first type console and two second type consoles if the number of patients to be diagnosed with the second type is greater.

According to a thirteenth aspect, the ultrasonic diagnostic apparatus system according to the twelfth aspect, is characterized in that the type of the console differs with the clinical department.

The ultrasonic diagnostic apparatus system according to the thirteenth aspect enables the type of the console connected to the ultrasonic diagnostic apparatus body to be changed according to the clinical department.

According to a fourteenth aspect, the ultrasonic diagnostic apparatus system according to the thirteenth aspect, is characterized in that the clinical departments are the internal medicine department/urological department and the cardiovascular department.

The ultrasonic diagnostic apparatus system according to the fourteenth aspect enables the type of the console connected to the ultrasonic diagnostic apparatus body to be changed according to whether the clinical department is the internal medicine department/urological department or the cardiovascular department.

According to a fifteenth aspect, the ultrasonic diagnostic apparatus system according to the thirteenth aspect, is characterized in that the clinical departments are the internal medicine department/urological department and the obstetrics department/gynecology department.

The ultrasonic diagnostic apparatus system according to the fifteenth aspect enables the type of the console connected to the ultrasonic diagnostic apparatus body to be changed according to whether the clinical department is the internal medicine department/urological department or the obstetrics department/gynecology department.

According to a sixteenth aspect, the ultrasonic diagnostic apparatus system according to the thirteenth aspect, is characterized in that the clinical departments are the internal medicine department/urological department, the cardiovascular department and the obstetrics department/gynecology department.

The ultrasonic diagnostic apparatus system according to the sixteenth aspect enables the type of the console connected to the ultrasonic diagnostic apparatus body to be changed according to whether the clinical department is the internal medicine department/urological department, the cardiovascular department or the obstetrics department/gynecology department.

According to a seventeenth aspect, the ultrasonic diagnostic apparatus system according to any of the twelfth through sixteenth aspects, is characterized in that the console is equipped with a wireless communication interface for wireless communication with the ultrasonic diagnostic apparatus body and the ultrasonic diagnostic apparatus body is equipped with a wireless communication interface for wireless communication with the console.

As the ultrasonic diagnostic apparatus system according to the seventeenth aspect is wirelessly connected, there is no need to engage or disengage connectors, and accordingly the console can be replaced quickly.

The ultrasonic diagnostic apparatus and the ultrasonic diagnostic apparatus system according to the invention can use a console which permits execution of a plurality of functions required in hospital with a single apparatus and is convenient to use.

The ultrasonic diagnostic apparatus and the ultrasonic diagnostic apparatus system according to the invention can be utilized for ultrasonic diagnosis in a hospital having a plurality of different departments of medicine.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram illustrating the configuration of an ultrasonic diagnostic apparatus pertaining to a first embodiment.

FIG. 2 is a block diagram illustrating the configuration of the ultrasonic diagnostic apparatus shown in FIG. 1 when used in the general abdominal operation mode.

FIG. 3 is a block diagram illustrating the configuration of the ultrasonic diagnostic apparatus shown in FIG. 1 when used in the cardiovascular operation mode.

FIG. 4 is a block diagram illustrating the configuration of the ultrasonic diagnostic apparatus shown in FIG. 1 when used in the obstetric/gynecological operation mode.

FIG. 5 is a flow chart that shows processing by the ultrasonic diagnostic apparatus body shown in FIG. 1.

FIG. 6 is a flow chart that shows processing by a console shown in FIG. 1.

FIG. 7 is a block diagram illustrating the configuration of the ultrasonic diagnostic apparatus system pertaining to a second embodiment.

FIG. 8 is a block diagram illustrating the configuration of the ultrasonic diagnostic apparatus system shown in FIG. 7 when two ultrasonic diagnostic apparatuses therein are used in the general abdominal operation mode and one ultrasonic diagnostic apparatus therein is used in the cardiovascular operation mode.

FIG. 9 is a block diagram illustrating the configuration of the ultrasonic diagnostic apparatus system shown in FIG. 7 when one ultrasonic diagnostic apparatus therein is used in the general abdominal operation mode and two ultrasonic diagnostic apparatuses therein are used in the cardiovascular operation mode.

### DETAILED DESCRIPTION OF THE INVENTION

The invention will be described in further detail below with reference to modes for implementation illustrated in drawings. Incidentally, this is nothing to limit the invention.

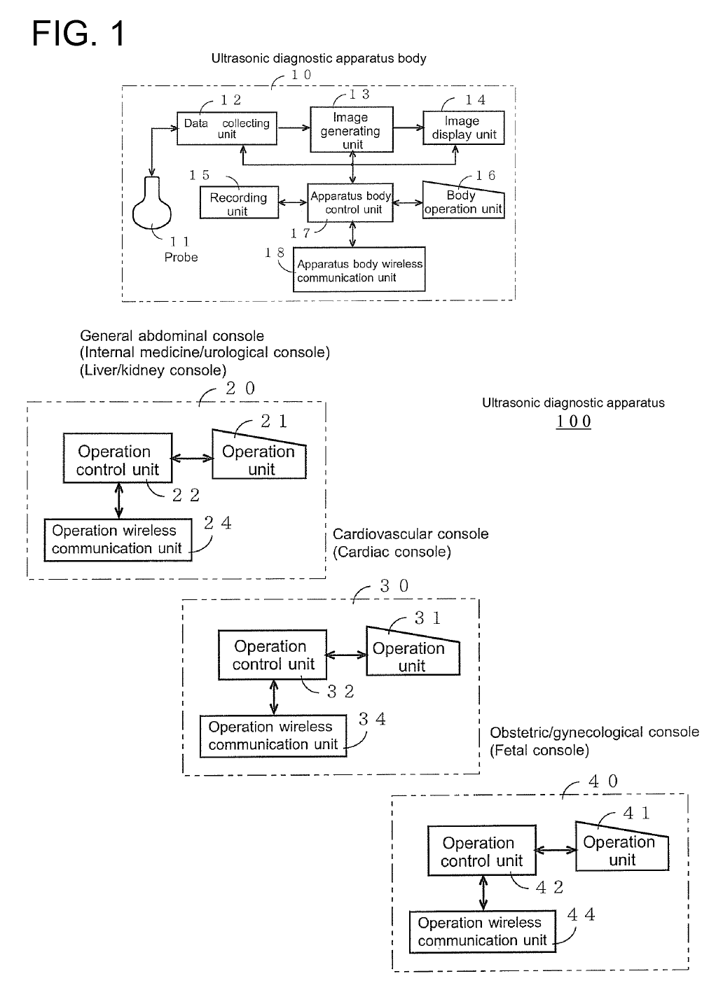

FIG. 1 is a diagram illustrating the configuration of an ultrasonic diagnostic apparatus 100 pertaining to a first embodiment.

This ultrasonic diagnostic apparatus 100 includes an ultrasonic diagnostic apparatus body 10, a general abdominal console 20, a cardiovascular console 30 and an obstetric/gynecological console 40, all separate from the ultrasonic diagnostic apparatus body 10.

Incidentally, the general abdominal console 20 is mainly used as an internal/urological console or a liver/kidney console. The cardiovascular console 30 is mainly used as a cardiac console. The obstetric/gynecological console 40 is mainly used as a fetal console.

The ultrasonic diagnostic apparatus body 10 includes: a body operation unit 16 for the operator to input instructions to; a probe 11; a data collecting unit 12 for driving the probe 11 to scan the inside of a subject with an ultrasonic beam and collect data; an apparatus body wireless communication unit 18 for receiving wirelessly instructions and data to the ultrasonic diagnostic apparatus body 10 from the consoles 20, 30 and 40 and transmitting wirelessly to consoles 20, 30 and 40 commands and messages addressed to the consoles 20, 30 and 40; an image generating unit 13 which generates ultrasonic images on the basis of the collected data; an image display unit 14 which displays ultrasonic images and the like; a recording unit 15 which records data, ultrasonic images and the like; and an apparatus body control unit 17 which controls the whole ultrasonic diagnostic apparatus body 10.

The general abdominal console 20 includes: an operation unit 21 for the operator to input instructions for ultrasonic diagnosis of the liver or the kidneys to; an operation wireless communication unit 24 for wireless communication with the ultrasonic diagnostic apparatus body 10; and an operation control unit 22 which controls the whole console 20.

The cardiovascular console 30 includes: an operation unit 31 for the operator to input instructions for ultrasonic diagnosis of the heart; an operation wireless communication unit 34 for wireless communication with the ultrasonic diagnostic apparatus body 10; and an operation control unit 32 which controls the whole console 30.

The obstetric/gynecological console 40 includes: an operation unit 41 for the operator to input instructions for ultrasonic diagnosis of the fetus; an operation wireless communication unit 44 for wireless communication with the ultrasonic diagnostic apparatus body 10; and an operation control unit 42 which controls the whole console 40.

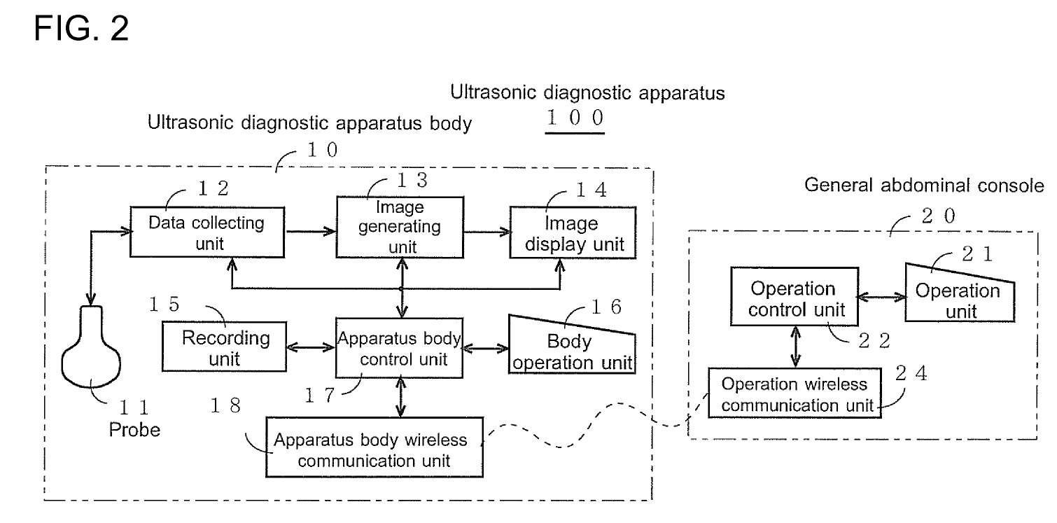

As shown in FIG. 2, when the general abdominal console 20 is connected to the ultrasonic diagnostic apparatus body 10, the ultrasonic diagnostic apparatus body 10 operates in a general abdominal operation mode which includes functions for the ultrasonic diagnosis of the liver and the kidneys.

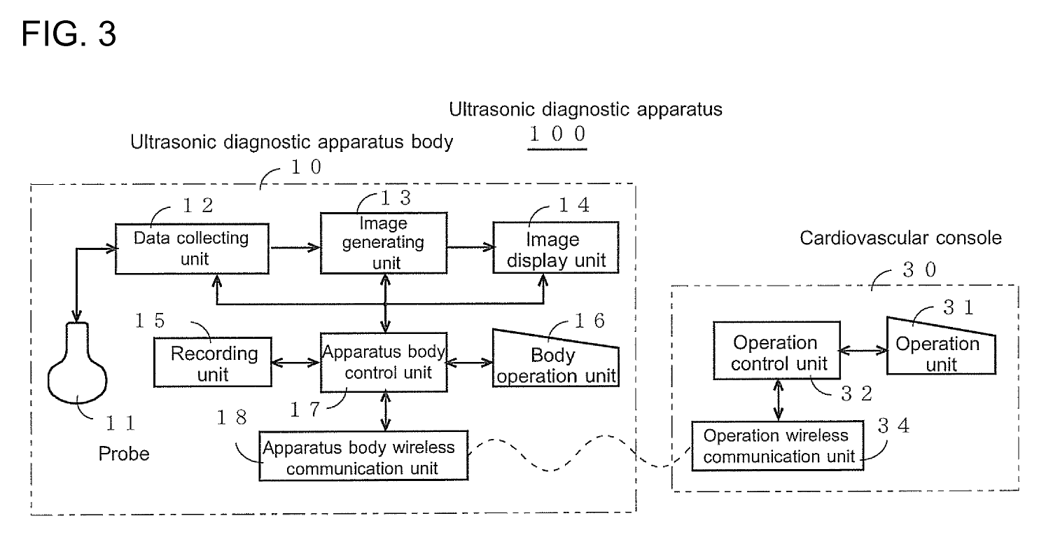

As shown in FIG. 3, when the cardiovascular console 30 is connected to the ultrasonic diagnostic apparatus body 10, the ultrasonic diagnostic apparatus body 10 operates in a cardiovascular operation mode that includes functions for the ultrasonic diagnosis of the heart.

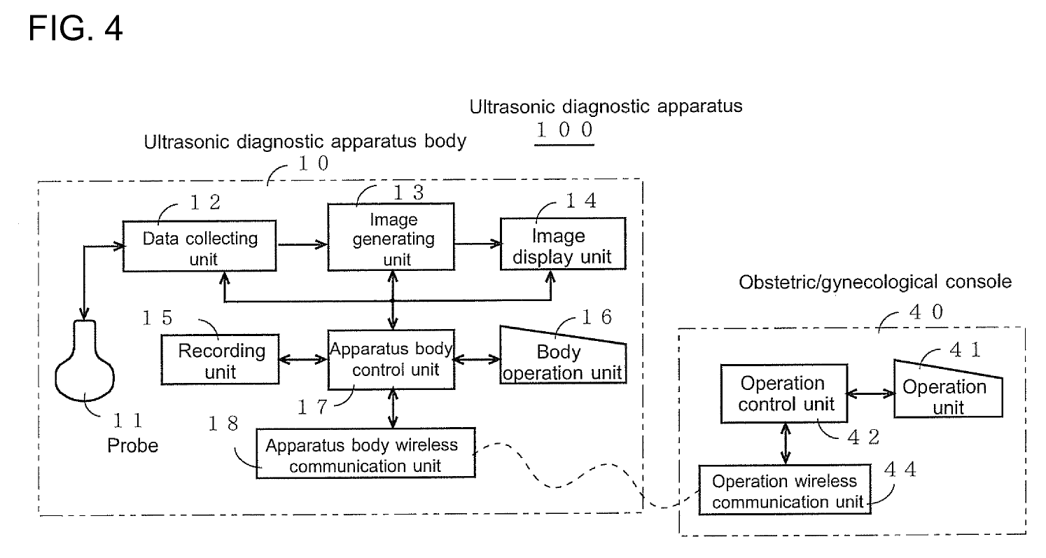

As shown in FIG. 4, when the obstetric/gynecological console 40 is connected to the ultrasonic diagnostic apparatus body 10, the ultrasonic diagnostic apparatus body 10 operates in an obstetric/gynecological operation mode which includes functions for the ultrasonic diagnosis of the fetus.

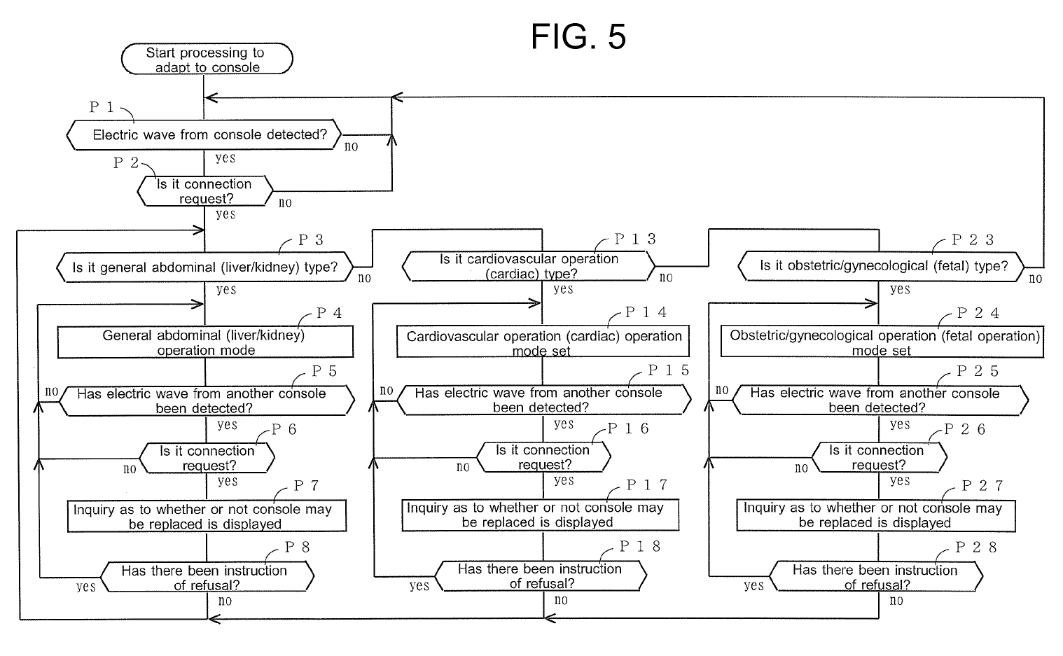

FIG. 5 is a flow chart that shows processing to adapt to the console executed by the ultrasonic diagnostic apparatus body 10.

At step P1, waiting takes place until an electric wave from the console 20, 30 or 40 is detected by the apparatus body wireless communication unit 18 and, upon detection the processing moves ahead to step P2.

At step P2, request type information contained in the electric wave from the console is checked and, if it is a “connection request”, the processing moves ahead to step P3 or, if not, returns to step P1.

At step P3, console type information contained in the electric wave from the console last detected is checked and, if it is a “general abdominal type”, the processing moves ahead to step P4 or, if not, moves ahead to step P1 P13.

At step P4, the operation takes place in the general abdominal operation mode.

At step P5, it is checked whether or not an electric wave from another console has been detected by the apparatus body wireless communication unit 18 and, if not, the processing returns to step P4 or, if any has been, the processing moves ahead to step P6.

At step P6, request type information contained in the electric wave from another console is checked and, if it is a “connection request”, the processing moves ahead to step P7 or, if not, returns to step P4.

At step P7, a message of inquiry as to whether or not the connected console may be replaced with another console is displayed on the image display unit 14.

At step P8, it is checked whether or not an instruction of refusal has been inputted from either the currently connected console or any other console having made detection and, if an instruction of refusal is inputted within a certain length of time, the processing returns to step P4 or, if no instruction of refusal is inputted within that length of time, the processing returns to step P3.

At step P13, console type information contained in the electric wave from the last detected console is checked and, if it indicates the “cardiovascular type”, the processing moves ahead to step P14 or, if not, moves ahead to step P23.

At step P14, the operation takes place in the cardiovascular operation mode.

At step P15, it is checked whether or not an electric wave from any other console has been detected by the apparatus body wireless communication unit 18 and, if not, the processing returns to step P14 or, if any has been, the processing moves ahead to step P16.

At step P16, request type information contained in the electric wave from the other console is checked and, if it is a “connection request”, the processing moves ahead to step P17 or, if not, returns to step P14.

At step P17, a message of inquiry as to whether or not the connected console may be replaced with another console is displayed on the image display unit 14.

At step P18, it is checked whether or not an instruction of refusal has been inputted from either the currently connected console or any other console having made detection and, if an instruction of refusal is inputted within a certain length of time, the processing returns to step P14 or, if no instruction of refusal is inputted within that length of time, the processing returns to step P3.

At step P23, console type information contained in the electric wave from the console last detected is checked and, if it is an “obstetric/gynecological type”, the processing moves ahead to step P24 or, if not, returns to step P1.

At step P24, the operation takes place in the obstetric/gynecological operation mode.

At step P25, it is checked whether or not an electric wave from any other console has been detected by the apparatus body wireless communication unit 18 and, if not, the processing returns to step P24 or, if any has been, the processing moves ahead to step P26.

At step P26, request type information contained in the electric wave from the other console is checked and, if it is a “connection request”, the processing moves ahead to step P27 or, if not, returns to step P24.

At step P27, a message of inquiry as to whether or not the connected console may be replaced with another console is displayed on the image display unit 14.

At step P28, it is checked whether or not an instruction of refusal has been inputted within a certain length of time (e.g. 10 seconds) from either the currently connected console or any other console having made detection and, if an instruction of refusal has been inputted, the processing returns to step P24 or, if no instruction of refusal has been inputted, the processing returns to step P3.

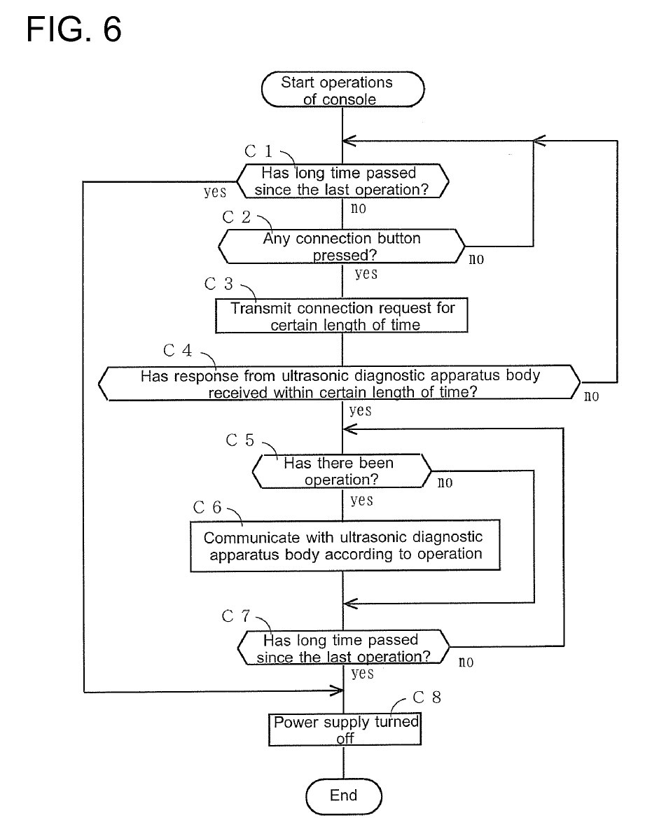

FIG. 6 is a flow chart showing the operations of the consoles 20, 30 and 40.

At step C1, it is checked whether or not a long time (e.g. one hour) has passed since the last inputting by the operator and, if not, the processing moves ahead to step C2 or, if a long time has passed, the processing moves ahead to step C8.

At step C2, it is checked whether or not the operator has pressed a connection button (included in the operation units 21, 31 and 41) and, if he has, the processing moves ahead to step C3 or, if not, the processing returns to step C1.

At step C3, transmission of an electric wave containing request type information and console type information is repeated for a certain length of time (e.g. three seconds).

At step C4, a response from the ultrasonic diagnostic apparatus body 10 is awaited and, if there is a response within a certain length of time (e.g. five seconds) the processing moves ahead to step C5 or, if there is no response, the processing returns to step C1.

At step C5, it is checked whether or not the operator has performed inputting and, if he has, the processing moves ahead to step C6 or, if not, the processing moves ahead to step C7.

At step C6, communication is performed with the ultrasonic diagnostic apparatus body 10 according to the inputting by the operator. Then, the processing moves ahead to step C7.

At step C7, it is checked whether or not a long time (e.g. one hour) has passed since the last inputting by the operator and, if not, the processing returns to step C5 or, if a long time has passed, the processing moves ahead to step C8.

At step C8, the power supply is turned off.

The ultrasonic diagnostic apparatus 100 of the first embodiment provides, but is not limited to providing, the following effects.

(1) By connecting the general abdominal console 20 to the ultrasonic diagnostic apparatus 100, functions mainly needed by the internal medicine department/urological department can be executed; by connecting the cardiovascular console 30, functions needed by the cardiovascular department can be executed; by connecting the obstetric/gynecological console 40, functions needed by the obstetrics department/gynecology department can be executed; and therefore the apparatus excels in cost effectiveness.

(2) Since the consoles 20, 30 and 40 specialized in operations for different functions can be used, there are no array of buttons or the like which are never used, and accordingly the console would be more convenient to use.

(3) Since the operation mode automatically changes according to the console connected, the workload on the operator is reduced.

(4) If doctor who are sent from universities to a hospital either regularly or irregularly carries with the consoles for their personal use and use them by connecting to the ultrasonic diagnostic apparatus body 10 available at the hospital, a plurality of doctors differing in the field of specialization can commonly use the ultrasonic diagnostic apparatus body 10, and the need to install ultrasonic diagnostic apparatuses each dedicated to a different department of medicine is reduced.

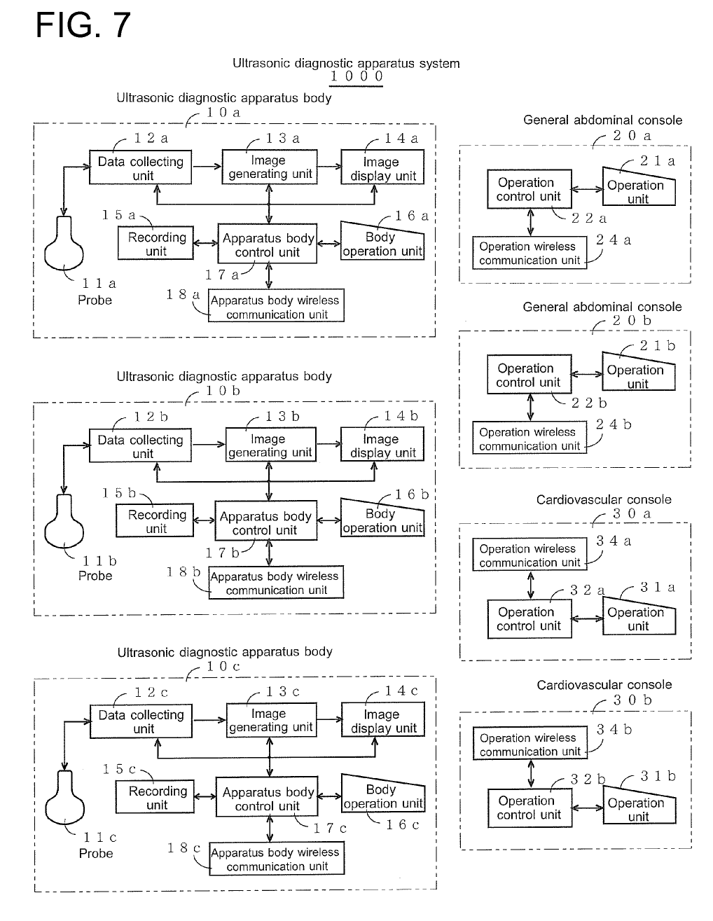

FIG. 7 is a diagram illustrating the configuration of an ultrasonic diagnostic apparatus system 1000 pertaining to a second embodiment.

This ultrasonic diagnostic apparatus system 1000 includes three ultrasonic diagnostic apparatus bodies 10a, 10b and 10c, two general abdominal consoles 20a and 20b, and two cardiovascular consoles 30a and 30b.

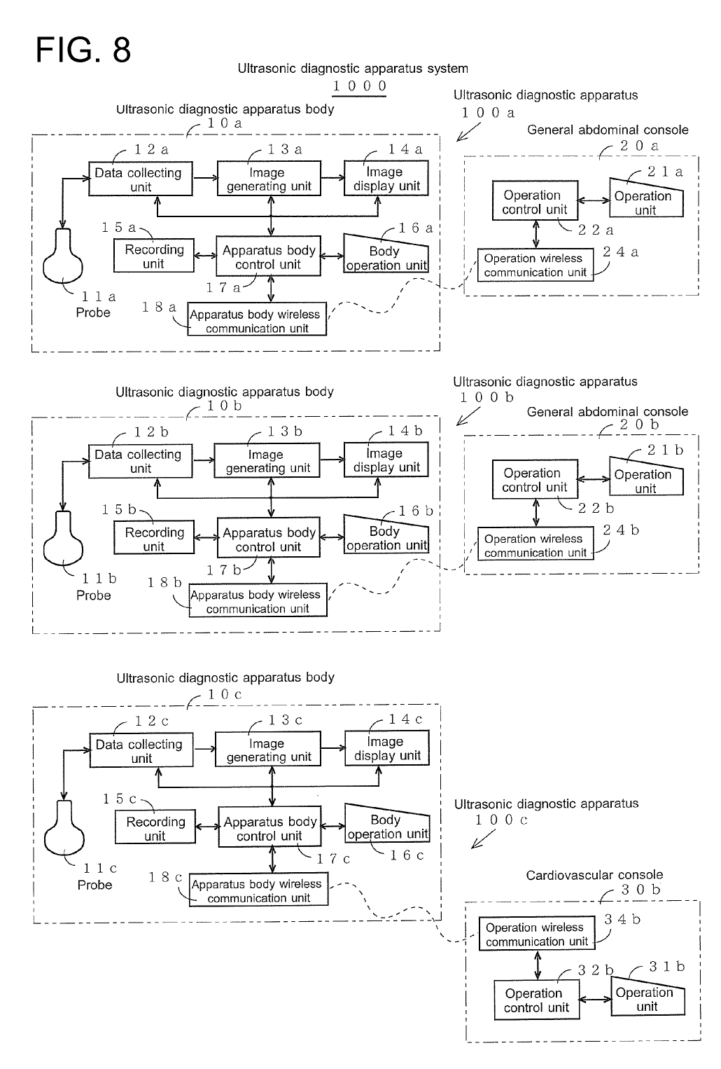

When the number of patients to be diagnosed in the general abdominal operation mode is greater than the number of patients to be diagnosed in the cardiovascular operation mode, the general abdominal console 20a is connected to the ultrasonic diagnostic apparatus body 10a, the general abdominal console 20b is connected to the ultrasonic diagnostic apparatus body 10b, and the cardiovascular console 30b is connected to the ultrasonic diagnostic apparatus body 10c as shown in FIG. 8. In this way, two ultrasonic diagnostic apparatuses 100a and 100b are operated in the general abdominal operation mode, and one ultrasonic diagnostic apparatus 100c is operated in the cardiovascular operation mode.

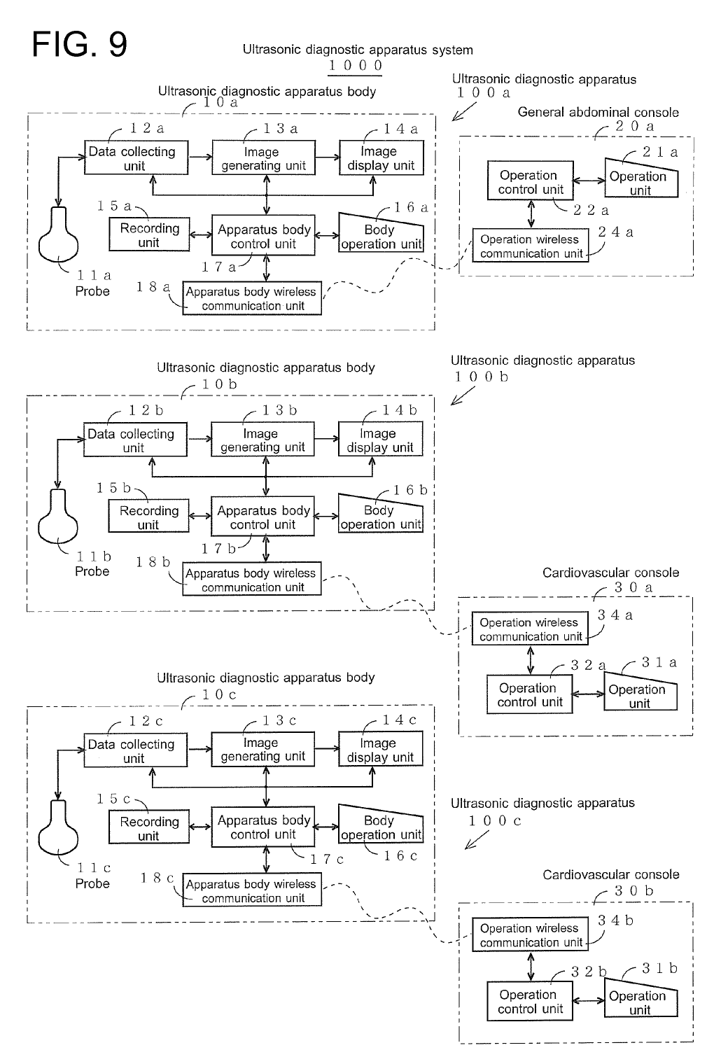

On the other hand, when the number of patients to be diagnosed in the cardiovascular operation mode is greater than the number of patients to be diagnosed in the general abdominal operation mode, the general abdominal console 20a is connected to the ultrasonic diagnostic apparatus body 10a, the cardiovascular console 30a is connected to the ultrasonic diagnostic apparatus body 10b, and the cardiovascular console 30b is connected to the ultrasonic diagnostic apparatus body 10c as shown in FIG. 9. In this way, one ultrasonic diagnostic apparatus 100a is operated in the general abdominal operation mode, and two ultrasonic diagnostic apparatuses 100b and 100c are operated in the cardiovascular operation mode.

The ultrasonic diagnostic apparatus system 1000 of the second embodiment enables the system configuration to be flexibly altered according to patients' needs.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a console of at least one of a plurality of different console types; and
an ultrasonic diagnostic apparatus body configured to be coupled to said console, said ultrasonic diagnostic apparatus body comprising a console interface device configured to permit changeable connection to each of the plurality of different types of consoles, a console identifying device configured to identify a type of connected console, and an operation mode control device configured to control a connected console in order to accomplish operation in the diagnostic mode corresponding to said connected console.

2. The ultrasonic diagnostic apparatus according to claim 1, wherein the type of connected console differs according to an object of diagnosis.

3. The ultrasonic diagnostic apparatus according to claim 2, wherein the object of diagnosis is one of a liver/kidney and a heart.

4. The ultrasonic diagnostic apparatus according to claim 2, wherein the object of diagnosis is one of a liver/kidney and a fetus.

5. The ultrasonic diagnostic apparatus according to claim 2, wherein the object of diagnosis is one of a liver/kidney, a heart, and a fetus.

6. The ultrasonic diagnostic apparatus according to claim 1, wherein said console comprises a wireless communication interface configured for wireless communication with said ultrasonic diagnostic apparatus body, and said ultrasonic diagnostic apparatus body further comprises a wireless communication interface configured for wireless communication with said console.

7. The ultrasonic diagnostic apparatus according to claim 2, wherein said console comprises a wireless communication interface configured for wireless communication with said ultrasonic diagnostic apparatus body, and said ultrasonic diagnostic apparatus body further comprises a wireless communication interface configured for wireless communication with said console.

8. The ultrasonic diagnostic apparatus according to claim 3, wherein said console comprises a wireless communication interface configured for wireless communication with said ultrasonic diagnostic apparatus body, and said ultrasonic diagnostic apparatus body further comprises a wireless communication interface configured for wireless communication with said console.

9. The ultrasonic diagnostic apparatus according to claim 4, wherein said console comprises a wireless communication interface configured for wireless communication with said ultrasonic diagnostic apparatus body, and said ultrasonic diagnostic apparatus body further comprises a wireless communication interface configured for wireless communication with said console.

10. The ultrasonic diagnostic apparatus according to claim 1, wherein the type of connected console differs according to a clinical department.

11. The ultrasonic diagnostic apparatus according to claim 10, wherein the clinical department is one of an internal medicine department/urological department and a cardiovascular department.

12. The ultrasonic diagnostic apparatus according to claim 10, wherein the clinical department is one of an internal medicine department/urological department and an obstetrics department/gynecology department.

13. The ultrasonic diagnostic apparatus according to claim 10, wherein the clinical department is one of an internal medicine department/urological department, a cardiovascular department, and an obstetrics department/gynecology department.

14. The ultrasonic diagnostic apparatus according to claim 10, wherein said console comprises a wireless communication interface configured for wireless communication with said ultrasonic diagnostic apparatus body, and the said ultrasonic diagnostic apparatus body further comprises a wireless communication interface configured for wireless communication with said console.

15. An ultrasonic diagnostic apparatus system comprising:
a plurality of consoles of at least two types of a plurality of different console types; and
a plurality of ultrasonic diagnostic apparatus bodies, each ultrasonic diagnostic apparatus body configured to be coupled to one of said plurality of consoles, each of said ultrasonic diagnostic apparatus body comprising a console interface device configured to permit changeable connection to each of the plurality of of different console types, a console identifying device configured to identify a type of connected console, and an operation mode control device configured to control a connected console in order to accomplish operation in a diagnostic mode corresponding to said connected console, wherein a number of consoles of said plurality of consoles is greater than a number of ultrasonic diagnostic apparatus bodies of said plurality of ultrasonic diagnostic apparatus bodies.

16. The ultrasonic diagnostic apparatus system according to claim 15, wherein the type of connected console differs according to a clinical department.

17. The ultrasonic diagnostic apparatus system according to claim 16, wherein the clinical department is one of an internal medicine department/urological department and a cardiovascular department.

18. The ultrasonic diagnostic apparatus system according to claim 16, wherein the clinical department is one of an internal medicine department/urological department and an obstetrics department/gynecology department.

19. The ultrasonic diagnostic apparatus system according to claim 16, wherein the clinical department is one of an internal medicine department/urological department, a cardiovascular department, and an obstetrics department/gynecology department.

20. The ultrasonic diagnostic apparatus system according to claim 15, wherein each of said plurality of consoles comprises a wireless communication interface configured for wireless communication with one of said plurality of ultrasonic diagnostic apparatus bodies, and wherein each of said plurality of ultrasonic diagnostic apparatus bodies further comprises a wireless communication interface configured for wireless communication with one of said plurality of consoles.
